(19) United States
(12) Patent Application Publication
(10) Pub. No.: US 2018/0085104 A1
(43) Pub. Date: Mar. 29, 2018

(54) ARTHROSCOPIC RETRACTING PROBE

(71) Applicant: Arthrex, Inc., Naples, FL (US)

(72) Inventors: Reinhold Schmieding, Naples, FL (US); Gary Scott Sherman, Naples, FL (US); Karen Gallen, Naples, FL (US); Andrew K. Osika, Naples, FL (US)

(21) Appl. No.: 15/277,154

(22) Filed: Sep. 27, 2016

(51) Int. Cl. A61B 17/02

(52) U.S. Cl. CPC A61B 17/025; A61B 17/0218

(57) ABSTRACT

A retracting probing instrument includes an outer tube having a longitudinal axis. The outer tube includes an inner passage and a tip. A probe includes an inner shaft having a distal end portion. At least a portion of the distal end portion of the inner shaft is made of nitinol. In an initial position, the distal end portion of the inner shaft is located in the inner passage of the outer tube. In a deployed position, the distal end portion of the inner shaft protrudes from the tip of the outer tube.

### BACKGROUND

This disclosure relates to an arthroscopic retracting probing instrument including a probe having a distal end portion, and at least a portion of the distal end portion is made of nitinol. When deployed from an outer tube, the distal end portion of the probe moves from an initial position to a deployed position.

For small joint arthroscopy or in office arthroscopy, there is a need for a small instrument that can be used easily and comfortably inserted into a small incision and a joint. A probe of the instrument must be small and stiff enough to palpate within the joint. The probe should also be retractable to be easily inserted into the joint.

### SUMMARY

In an embodiment, a retracting probing instrument includes an outer tube having a longitudinal axis. The outer tube includes an inner passage and a tip. A probe includes an inner shaft having a distal end portion. At least a portion of the distal end portion of the inner shaft is made of nitinol. In an initial position, the distal end portion of the inner shaft is located in the inner passage of the outer tube. In a deployed position, the distal end portion of the inner shaft protrudes from the tip of the outer tube.

In another embodiment, an entirety of the inner shaft is made of nitinol.

In another embodiment according to any of the previous embodiments, the distal end portion of the inner shaft includes a tip portion, and the tip portion is made of nitinol.

In another embodiment according to any of the previous embodiments, the outer tube is substantially straight, and the tip has an angled surface.

In another embodiment according to any of the previous embodiments, the outer tube has an outer diameter of approximately 0.065+/−0.0005 inches and an inner diameter of approximately 0.047+/−0.0015 inches. In another embodiment according to any of the previous embodiments, the outer tube has an outer diameter of approximately 0.083+/−0.0015 inches and an inner diameter of approximately 0.063+/−0.0015 inches.

In another embodiment according to any of the previous embodiments, the inner shaft is a wire that has a circular cross-section.

In another embodiment according to any of the previous embodiments, a diameter of the wire is approximately 0.039 to 0.042 inches.

In another embodiment according to any of the previous embodiments, the inner shaft is a wire that has a substantially rectangular cross-section.

In another embodiment according to any of the previous embodiments, the substantially rectangular cross-section of the wire has a first dimension of approximately 0.04 inches and a second dimension of approximately 0.01 inches.

In another embodiment according to any of the previous embodiments, the inner shaft is a wire and the outer tube is a needle, and the wire includes a proximal substantially straight portion, a distal substantially straight portion, and a curved portion located therebetween. In an initial position, the distal substantially straight portion is located inside the needle. In a deployed position, the distal substantially straight portion is located outside the needle and extends approximately 90° relative to the proximal substantially straight portion.

In another embodiment according to any of the previous embodiments, the distal end portion of the wire extends approximately 3.0 mm+/−2.0 mm from the tip of the needle when in the deployed position.

In another embodiment according to any of the previous embodiments, the outer tube includes a first locking feature and the probe includes a second locking feature that interacts with the first locking feature to secure the probe to the outer tube in the initial position.

In another embodiment according to any of the previous embodiments, a distal end of the probe includes a rotating probe tip.

In another embodiment according to any of the previous embodiments, the rotating probe tip has a diameter of approximately 2 mm.

In another embodiment according to any of the previous embodiments, the rotating probe tip moves between the initial position substantially parallel with the inner shaft and the deployed position substantially perpendicular to the inner shaft.

In another embodiment according to any of the previous embodiments, the retracting probing instrument includes a handle, and the inner shaft has an area of reduced diameter that is located proximate to the handle.

In another embodiment according to any of the previous embodiments, a probing instrument includes a handle and an outer tube that is substantially straight and includes an inner passage and a longitudinal axis. The probing instrument includes an inner shaft within the inner passage of the outer tube having a longitudinal axis parallel to the longitudinal axis of the outer tube, and the inner shaft comprises a break point proximal to the handle. The probing instrument also includes a rotating probe tip at distal ends of the outer tube and the inner shaft, and the rotating probe tip is configured to rotate from a first position generally aligned with a longitudinal axes of the outer tube and the inner shaft to a second position which is not aligned with the longitudinal axes. The outer tube allows the rotating probe tip to pivot from an angle of about zero degrees relative to the longitudinal axis of the outer tube to an angle of about ninety degrees relative to the longitudinal axis of the tube.

In another embodiment according to any of the previous embodiments, the outer tube includes a first locking feature and the inner shaft includes a second locking feature that interacts with the first locking feature to secure the inner shaft to the outer tube in an initial position.

In another embodiment according to any of the previous embodiments, the rotating probe tip comprises nitinol.

In another embodiment according to any of the previous embodiments, the probing instrument includes a mechanism comprising a pin and a slot that allow conversion of linear movement of the inner shaft into rotational movement of the rotating probe tip to the second position upon linear movement of the inner shaft in relation to the outer tube. The pin slides in the slot to permit rotation of the rotating probe tip, and the mechanism locks the rotating probe tip on the outer tube when the rotating probe tip is in the second position.

### DETAILED DESCRIPTION OF THE DRAWINGS

FIG. 1 illustrates a side view of a first example arthroscopic retracting probing instrument;

FIG. 2 illustrates a circular cross section of a wire of a probe;

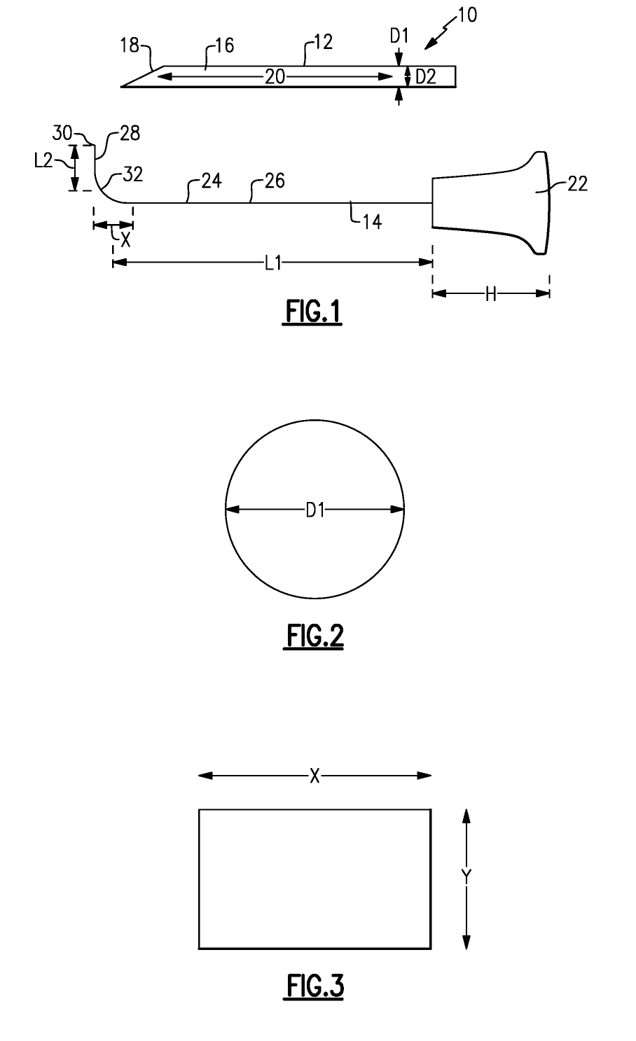

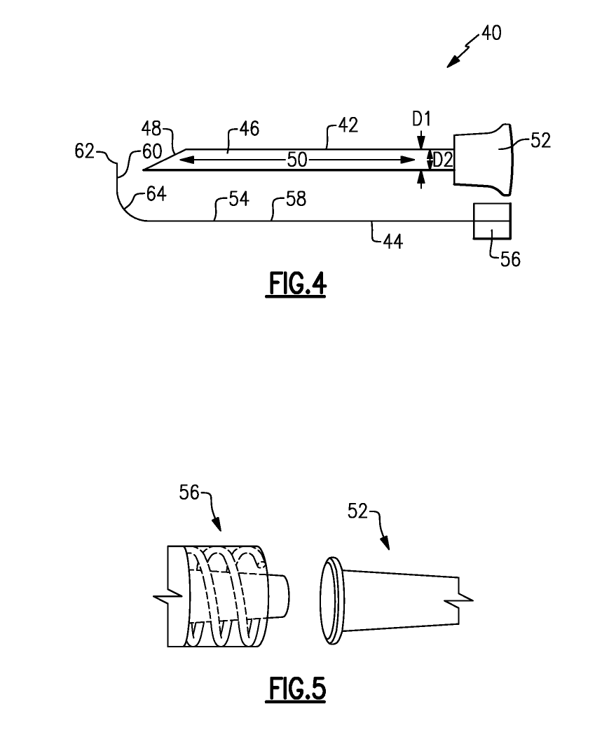

FIG. 3 illustrates a rectangular cross section of another example wire of the probe;

FIG. 4 illustrates a second example arthroscopic retracting probing instrument;

FIG. 5 illustrates a first locking feature and a second locking feature of a needle and a probe of the second example arthroscopic retracting probing instrument of FIG. 4;

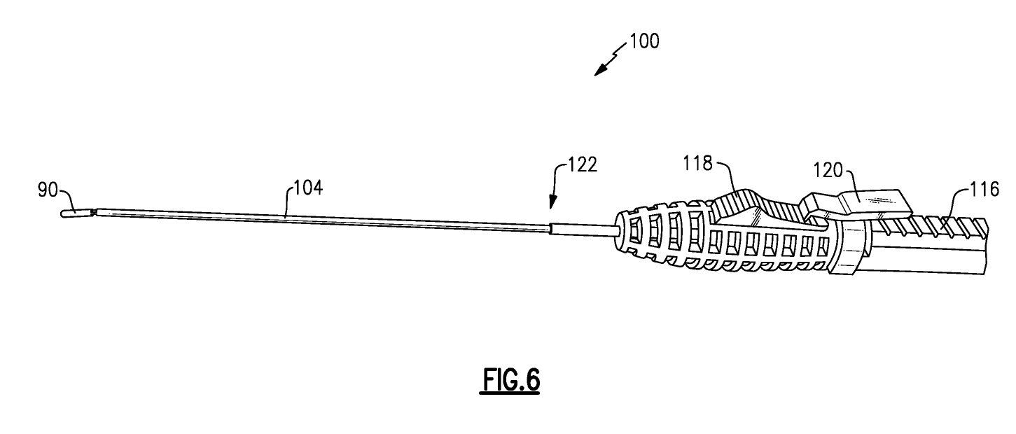

FIG. 6 illustrates the third example arthroscopic retracting probe instrument;

FIG. 7 illustrates a rotating probe tip of the retracting probe instrument of FIG. 6 in an initial position;

FIG. 8 illustrates the retracting probe tip of the retracting probe instrument of FIG. 6 in a sequential “flip” position;

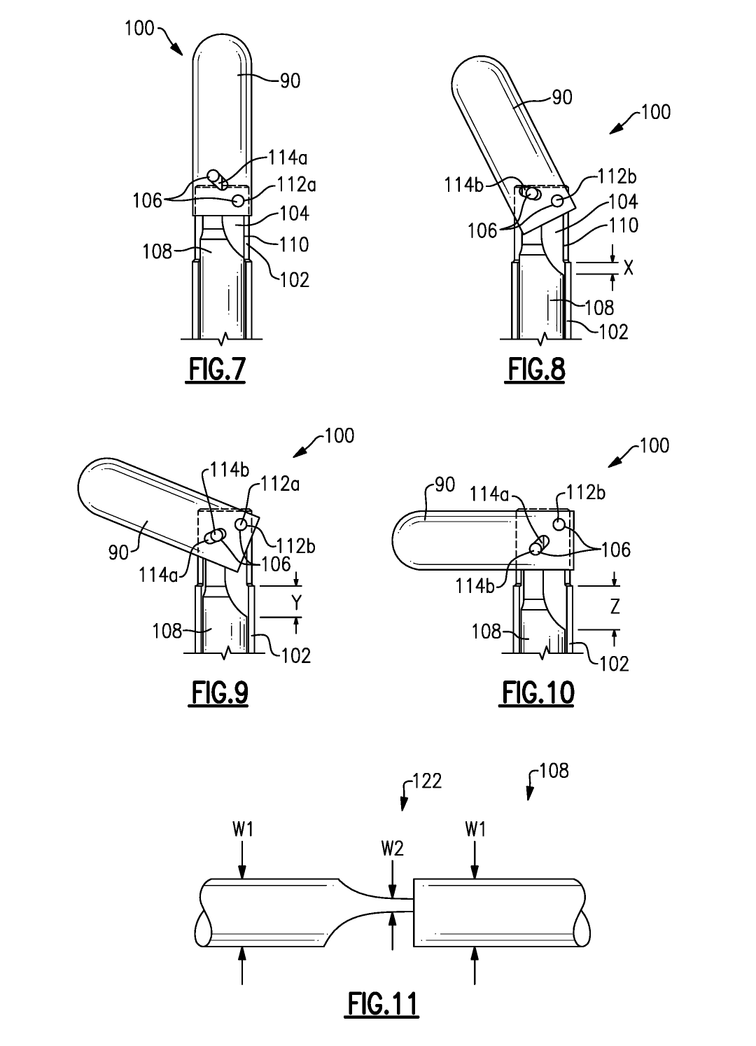

FIG. 9 illustrates the retracting probe tip of the retracting probe instrument of FIG. 6 in another sequential “flip” position;

FIG. 10 illustrates the retracting probe tip of the retracting probe instrument of FIG. 6 in the deployed position or “flip” position; and

FIG. 11 illustrates an inner shaft of the retracting probe instrument of FIG. 6.

### DETAILED DESCRIPTION

In an embodiment, a retracting probing instrument includes an outer tube having a longitudinal axis. The outer tube includes an inner passage and a tip. A probe includes an inner shaft having a distal end portion. At least a portion of the distal end portion of the inner shaft is made of nitinol. In an initial position, the distal end portion of the inner shaft is located in the inner passage of the outer tube. In a deployed position, the distal end portion of the inner shaft protrudes from the tip of the outer tube.

In another embodiment, an entirety of the inner shaft is made of nitinol.

In another embodiment according to any of the previous embodiments, the distal end portion of the inner shaft includes a tip portion, and the tip portion is made of nitinol.

In another embodiment according to any of the previous embodiments, the outer tube is substantially straight, and the tip has an angled surface.

In another embodiment according to any of the previous embodiments, the outer tube has an outer diameter of approximately 0.065+/−0.0005 inches and an inner diameter of approximately 0.047+/−0.0015 inches. In another embodiment according to any of the previous embodiments, the outer tube has an outer diameter of approximately 0.083+/−0.0015 inches and an inner diameter of approximately 0.063+/−0.0015 inches.

In another embodiment according to any of the previous embodiments, the inner shaft is a wire that has a circular cross-section.

In another embodiment according to any of the previous embodiments, a diameter of the wire is approximately 0.039 to 0.042 inches.

In another embodiment according to any of the previous embodiments, the inner shaft is a wire that has a substantially rectangular cross-section.

In another embodiment according to any of the previous embodiments, the substantially rectangular cross-section of the wire has a first dimension of approximately 0.04 inches and a second dimension of approximately 0.01 inches.

In another embodiment according to any of the previous embodiments, the inner shaft is a wire and the outer tube is a needle, and the wire includes a proximal substantially straight portion, a distal substantially straight portion, and a curved portion located therebetween. In an initial position, the distal substantially straight portion is located inside the needle. In a deployed position, the distal substantially straight portion is located outside the needle and extends approximately 90° relative to the proximal substantially straight portion.

In another embodiment according to any of the previous embodiments, the distal end portion of the wire extends approximately 3.0 mm+/−2.0 mm from the tip of the needle when in the deployed position.

In another embodiment according to any of the previous embodiments, the outer tube includes a first locking feature and the probe includes a second locking feature that interacts with the first locking feature to secure the probe to the outer tube in the initial position.

In another embodiment according to any of the previous embodiments, a distal end of the probe includes a rotating probe tip.

In another embodiment according to any of the previous embodiments, the rotating probe tip has a diameter of approximately 2 mm.

In another embodiment according to any of the previous embodiments, the rotating probe tip moves between the initial position substantially parallel with the inner shaft and the deployed position substantially perpendicular to the inner shaft.

In another embodiment according to any of the previous embodiments, the retracting probing instrument includes a handle, and the inner shaft has an area of reduced diameter that is located proximate to the handle.

In another embodiment according to any of the previous embodiments, a probing instrument includes a handle and an outer tube that is substantially straight and includes an inner passage and a longitudinal axis. The probing instrument includes an inner shaft within the inner passage of the outer tube having a longitudinal axis parallel to the longitudinal axis of the outer tube, and the inner shaft comprises a break point proximal to the handle. The probing instrument also includes a rotating probe tip at distal ends of the outer tube and the inner shaft, and the rotating probe tip is configured to rotate from a first position generally aligned with a longitudinal axes of the outer tube and the inner shaft to a second position which is not aligned with the longitudinal axes.

In another embodiment according to any of the previous embodiments, the outer tube includes a first locking feature and the inner shaft includes a second locking feature that interacts with the first locking feature to secure the inner shaft to the outer tube in an initial position.

In another embodiment according to any of the previous embodiments, the rotating probe tip comprises nitinol.

In another embodiment according to any of the previous embodiments, the probing instrument includes a mechanism comprising a pin and a slot that allow conversion of linear movement of the inner shaft into rotational movement of the rotating probe tip to the second position upon linear movement of the inner shaft in relation to the outer tube. The pin slides in the slot to permit rotation of the rotating probe tip, and the mechanism locks the rotating probe tip on the outer tube when the rotating probe tip is in the second position.

The tip of the outer tube can be inserted into a small incision or a joint. Once inserted, the probe can be disengaged from the outer tube. When the probe deploys, a distal portion of the outer tube protrudes from the tip of the outer tube and curves approximately 90° relative to the outer tube. When the probe is no longer needed, the probe is retracted back into the outer tube, and the outer tube is removed from the small incision.

A probing instrument as disclosed herein can be used during arthroscopy procedures. For example, the probing instrument's size allows its use during in office arthroscopy procedures. A physician could utilize the instrument to probe a knee or shoulder joint in an office visit. Further, the probing instrument's size allows its use during small joint (e.g., hand, wrist, foot, and ankle) arthroscopy procedures, whether in an office visit or in an operating room.

A method of arthroscopy includes stabbing the skin of a subject (i.e., a stab incision) with a probing instrument as described herein and deploying the retractable probe. In an embodiment, a probing instrument as described herein can be inserted into an incision and followed with deployment of the retractable probe.

Once the inner shaft is deployed, methods further including probing a joint. A user can probe a joint for loose bodies, defects (e.g., in cartilage), damage (e.g., cartilage damage), abnormalities, overall subject anatomy, bone spurs, etc.

FIG. 1 illustrates an arthroscopic retracting probing instrument 10 employed in small joint arthroscopy or in office arthroscopy. The arthroscopic retracting probing instrument 10 includes a hollow needle 12 and a retractable probe 14.

In one example, the needle 12 is a 14 gauge or a 16 gauge spinal needle. The needle 12 is substantially straight and has a longitudinal axis 20. The needle 12 includes an inner passage 15 and a tip 18 having an angled surface at a distal end of the needle 12. A 16 gauge needle 12 has an outer diameter D1 of approximately 0.065+/−0.0005 inches and an inner diameter D1 of approximately 0.047+/−0.0015 inches. A 14 gauge needle 12 has an outer diameter D1 of approximately 0.083+/−0.0005 inches and an inner diameter D2 of 0.063+/−0.0015 inches.

The probe 14 includes a wire 24 received inside the inner passage 16 of the needle 12. The wire 24 made entirely of nitinol or made of a material and has a nitinol tip. The probe 14 also includes a handle 22, and the wire 24 is attached to the handle 22. The wire 24 includes a proximal substantially straight portion 26 connected to the handle 22, a distal substantially straight portion 28 that includes a blunt tip 30, and a connection portion or curved portion 32 located between the proximal substantially straight portion 26 and the distal substantially straight portion 28. In one example, the proximal substantially straight portion 26 has a length L1 of approximately 7.00 inches, and the distal substantially straight portion 28 has a length L2 of approximately 3 mm and extends approximately 90° from the proximal substantially straight portion 26. The wire 24 is stiff, but flexible enough to be straightened to be received in the inner passage 16 of the straight needle 12. The wire 24 has a tight curve that causes the distal substantially straight portion 28 of the wire 24 to flex 90° when removed from the needle 12.

In one example, the handle 22 is made of plastic. The handle 22 secures the probe 14 to the needle 12, and the handle 22 has a length H of approximately 0.8 inches taken parallel to the longitudinal axis 20 of the needle 12.

In one example shown in FIG. 2, the wire 24 has a circular cross section. In this example, the diameter of the wire 24 is approximately 0.039 to 0.042 inches. In another example, the diameter is 0.031 inches. In another example shown in FIG. 3, the wire 24 has a rectangular cross section. In this example, the wire 24 has a dimension X of approximately 0.04 inches and a dimension Y of approximately 0.01 inches.

When the probe 14 is inside the inner passage 16 of the needle 12, the probe 14 is in an initial position. Although the probe 14 includes the curved portion 32, the needle 12 prevents the wire 24 from curving. After the needle 12 is inserted into the joint, the handle 22 of the probe 14 is pushed, allowing the wire 24 to be deployed and protrude through the tip 18 of the needle 12 such that the probe 14 moves to the extended position. As the curved portion 32 of the probe 14 exits the needle 12, the needle 12 no longer constrains the curved portion 32, allowing the distal substantially straight portion 28 to extend approximately 90° relative to the longitudinal axis 20 of the needle 12. The wire 24 extends a distance X from the needle 12 when in the deployed position. In one example, the distance X is 3.0 mm+/−2.0 mm. The wire 24 is then located in a joint to perform the procedure.

FIG. 4 illustrates an arthroscopic retracting probing instrument 40 including a needle 42 and a probe 44. In this example, the needle 42 is a needle of the Suture Lasso™, manufactured by Arthrex, Inc. of Naples, Fla. The needle 42 is substantially straight and includes an inner passage 46, a tip 48 having an angled surface at a distal end, and a longitudinal axis 50. The inner passage 46 tapers from a proximal end to a distal end of the needle 42. The needle 42 has an outer diameter D1 of approximately 0.065+/−0.0005 inches and an inner diameter D2 of 0.047+/−0.0015 inches. The needle 42 includes a first locking feature 52.

In one example, the probe 44 includes a wire 54 made entirely of nitinol. In another example, the wire 54 is made of a material and has a nitinol tip. The wire 54 has a diameter of approximately 0.04 inches. The probe 44 also includes a second locking feature 56.

When the probe 44 is received in the needle 42, the first locking feature 52 and the second locking feature 56 interact to secure the probe 44 relative to the needle 42. As shown in FIG. 5, both the first locking feature 52 and the second locking feature 56 define a luer lock connection, as known. However, the first locking feature 52 and the second locking feature 56 can be any type of locking features that secure the probe 44 to the needle 42.

The arthroscopic retracting probe 40 includes many of the same features as the arthroscopic retracting probe instrument 10. When the wire 54 is inside the inner passage 16 of the needle 12, the probe 44 is in an initial position. The probe 44 includes a curved portion 64 (located between a proximal substantially straight portion 58 and a distal substantially straight portion 60 having a blunt tip 62), but the wire 54 is prevented from curving when inside the inner passage 46 of the needle 12. After the needle 42 is inserted into the joint, the first locking feature 52 and the second locking feature 56 are disengaged, allowing the wire 54 to be deployed through the tip 48 of the needle 42 and move to the deployed position. The second locking feature 56 of the probe 44 is rotated relative to the first locking feature 52 of the needle 42 to deploy the probe 44. As the curved portion 64 of the probe 44 exits the needle 42, the needle 42 no longer constrains the curved portion 64, allowing the distal substantially straight portion 60 to extend approximately 90° relative to the longitudinal axis 50 of the needle 42. The wire 54 extends a distance X of 3.0 mm+/−2.0 mm from the needle 42 when in the deployed position. The wire 54 can be inserted into a joint to perform the procedure.

FIG. 6 illustrates another example arthroscopic retracting probing instrument 100 including a rotating probe tip 90. At least a portion of the rotating probe tip 90 is made of nitinol. In one example, the entire rotating probe tip 90 is made of nitinol. In one example, the rotating probe tip 90 has a diameter of about 2 mm.

As shown in FIGS. 7 to 10, the arthroscopic retracting probing instrument 100 includes a cannulated elongated outer tube 102 having a distal end 104 and a proximal end (not shown). The distal end 104 includes (at the most distal part) a mechanism 106 configured to engage the rotating probe tip 90 attached and securely engaged to the outer tube 102.

The outer tube 102 of the probe 99 houses an inner shaft 108 having a diameter smaller W1 than the diameter of the outer tube 102. The rotating probe tip 90 is provided at the distal end 104 of the outer tube 102 and is connected to both the outer tube 102 and the inner shaft 108 by the mechanism 106. In one embodiment, the rotating probe tip 90 is pinned to the outer tube 102 and the inner shaft 108. The outer tube 102 is provided with a cutout 110 that allows the rotating probe tip 90 to move within the cutout 110 and relative to the outer tube 102. The rotating probe tip 90 may have a body provided in various shapes and geometries.

The mechanism 106 includes a pin and a slot that allow conversion of the linear movement of the inner shaft 108 into rotational movement of the rotating probe tip 90. In one example, the mechanism 106 includes a first pin hole 112a (or first pin slot 112a) with a first pin 112b connecting the rotating probe tip 90 to the outer tube 102, and the first pin hole 112a permits only rotational movement. The mechanism 106 includes a second pin hole 114a (or second pin slot 114a) with a second pin 114b connecting the rotating probe tip 90 to inner shaft 108, where the second pin hole 114a is a slot permitting rotational and sliding movement of the rotating probe tip 90 relative to the second pin 114b.

As shown in FIGS. 8 and 9, when the outer tube 102 is advanced in a linear direction parallel to the longitudinal axis of the arthroscopic retracting probing instrument 100, the first pin 112b pushes one side of the proximal end of the rotating probe tip 90 in the linear direction, while the second pin 114b is permitted to slide in the slot of the second pin hole 114a, permitting rotation of the rotating probe tip 90.

In use, the rotating probe tip 90 is attached to both the outer tube 102 and the inner shaft 108 by the mechanism 106. The outer tube 102 and the inner shaft 108, with the rotating probe tip 90 attached and locked in a “straight” configuration (or in the initial position), are inserted into a joint from a distal side until they are located in the joint. That is, the rotating probe tip 90 is substantially parallel to the longitudinal axis of the arthroscopic retracting probing instrument 100.

Once the arthroscopic retracting probing instrument 100 is inserted in the joint, a linear motion may be carried out so that one of the outer tube 102 and the inner shaft 108 advances relative to the other of the outer tube 102 and the inner shaft 108 (for example, the outer tube 102 advances relative to the inner shaft 108) by a sequential distances x (FIG. 8), y (FIG. 9) and z (FIG. 10). At the point where the outer tube 102 travels the distance z relative to the inner shaft 108 (or when the inner shaft 108 travels the distance z relative to the outer tube 102), the rotating probe tip 90 is in a deployed position, or a locked or “flip” position. In the deployed position, the rotating probe tip 90 is substantially perpendicular to the longitudinal axis of the arthroscopic retracting probing instrument 100. That is, the rotating probe tip 90 moves approximately 90°. Movement of the outer tube 102 relative to the inner shaft 108 (i.e., while traveling a distance between about 0 to about z) converts the linear motion of the outer tube 102 into a rotational motion of the rotating probe tip 90. The arthroscopic retracting probing instrument 100 can then be used in an arthroscopic procedure or an in office procedure.

FIGS. 7 to 10 illustrate the outer tube 102 being moved linearly. However, it should be understood that the articulation of the rotating probe tip 90 to the deployed position occurs according to relational movement of the outer tube 102 and the inner shaft 108. Therefore, other embodiments could include the inner tube 108 being moved in a linear (distal or proximal) direction.

As further shown in FIG. 6, the arthroscopic retracting probing instrument 100 includes a handle 116 with a push button mechanism 118 that is pressed in a distal direction for deployment of the rotating probe tip 90 from the initial position to the deployed position. The push button mechanism 118 advances the outer tube 102 or the inner shaft 108 in a linear direction (for example, a distal direction) to rotate the rotating probe tip 90 to the deployed position or “flip” position. A locking ring 120 is slid in the distal direction to lock the push button mechanism 118 and therefore secure the rotating probe tip 90 in the deployed position. When the rotating probe tip 90 is to return to the initial position, the locking ring 120 is slid in a proximal direction, allowing the push button mechanism 118 to be moved in the proximal direction to allow the rotating probe tip 90 to return to the initial position.

As shown in FIG. 11, the arthroscopic retracting probing instrument 100 also includes break point 122 proximate to the handle 116. The diameter of the inner shaft 108 is W1. At the break point 122, the diameter of the inner shaft 108 narrows to W2. That is, the diameter W2 is less than the diameter W1. This creates a location of weakness at the location of W2. At the location of the smaller diameter portion W2, the inner shaft 108 has a lower strength than the strength of the rotating probe tip 90. If a breakage occurs when the arthroscopic retracting probing instrument 100 is in use, the breakage will occur at the break point 122 at the location of W2 and will not occur at or near the rotating probe tip 90. In the event of breakage, the instrument will break outside of a patient's body, and no part of the arthroscopic retracting probing instrument 100 will remain inside the body of the patient.

Although a preferred embodiment of this invention has been disclosed, a worker of ordinary skill in this art would recognize that certain modifications would come within the scope of this invention. For that reason, the following claims should be studied to determine the true scope and content of this invention.

What is claimed is:

1. A retracting probing instrument comprising:
an outer tube having a longitudinal axis, the outer tube including an inner passage and a tip; and
an inner shaft including a distal end portion, at least a portion of the distal end portion is made of nitinol, and the distal end portion of the inner shaft is located in the inner passage of the outer tube when in an initial position, and the distal end portion of the inner shaft protrudes from the tip of the outer tube in a deployed position.

2. The retracting probing instrument as recited in claim 1, wherein an entirety of the inner shaft is made of nitinol.

3. The retracting probing instrument as recited in claim 1, wherein the distal end portion of the inner shaft includes a tip portion, and the tip portion is made of nitinol.

4. The retracting probing instrument as recited in claim 1, wherein the outer tube is substantially straight, and the tip has an angled surface.

5. The retracting probing instrument as recited in claim 1, wherein the outer tube has one of a) an outer diameter of approximately 0.065 inches and an inner diameter of approximately 0.047+/−0.0015 and b) an outer diameter of approximately 0.083+/−0.0005 inches and an inner diameter of approximately 0.063+/−0.0015 inches.

6. The retracting probing instrument as recited in claim 1, wherein the inner shaft is a wire, and the wire has a circular cross-section.

7. The retracting probing instrument as recited in claim 6, wherein a diameter of the wire is approximately 0.039 inches to 0.042 inches.

8. The retracting probing instrument as recited in claim 1, wherein the inner shaft is a wire, and the wire has a substantially rectangular cross-section.

9. The retracting probing instrument as recited in claim 8, wherein the substantially rectangular cross-section of the wire has a first dimension of approximately 0.04 inches and a second dimension of approximately 0.01 inches.

10. The retracting probing instrument as recited in claim 1, wherein the inner shaft is a wire and the outer tube is a needle, and the wire includes a proximal substantially straight portion, a distal substantially straight portion, and a curved portion located therebetween, wherein the distal substantially straight portion is located inside the needle when in the initial position, and the distal substantially straight portion is located outside the needle and extends approximately 90° relative to the proximal substantially straight portion when in the deployed position.

11. The retracting probing instrument as recited in claim 10, wherein the distal end portion of the wire extends approximately 3 mm+/−2.0 mm from the tip of the needle when in the deployed position.

12. The retracting probing instrument as recited in claim 10, wherein the needle includes a first locking feature and the probe includes a second locking feature that interacts with the first locking feature to secure the probe to the needle in the initial position.

13. The retracting probing instrument as recited in claim 1, wherein a distal end of the probe includes a rotating probe tip.

14. The retracting probing instrument as recited in claim 13, wherein the rotating probe tip has a diameter of approximately 2 mm.

15. The retracting probing instrument as recited in claim 1, wherein the rotating probe tip moves between the initial position substantially parallel with the inner shaft and the deployed position substantially perpendicular to the inner shaft.

16. The retracting probing instrument as recited in claim 15, including a handle, wherein the inner shaft has an area of reduced diameter that is proximate to the handle.

17. A probing instrument comprising:
a handle;
an outer tube that is substantially straight and includes an inner passage and a longitudinal axis;
an inner shaft within the inner passage of the outer tube having a longitudinal axis parallel to the longitudinal axis of the outer tube, wherein the inner shaft comprises a break point proximal to the handle; and
a rotating probe tip at distal ends of the outer tube and the inner shaft, wherein the rotating probe tip is configured to rotate from a first position generally aligned with the longitudinal axes of the outer tube and the inner shaft to a second position which is not aligned with the longitudinal axes.

18. The probing instrument as recited in claim 17 wherein the outer tube includes a first locking feature and the inner shaft includes a second locking feature that interacts with the first locking feature to secure the inner shaft to the outer tube in an initial position.

19. The probing instrument as recited in claim 17 wherein the rotating probe tip comprises nitinol.

20. The probing instrument as recited in claim 17 further comprising a mechanism comprising a pin and a slot that allow conversion of linear movement of the inner shaft into rotational movement of the rotating probe tip to the second position upon linear movement of the inner shaft in relation to the outer tube, the pin sliding in the slot to permit rotation of the rotating probe tip, wherein the mechanism locks the rotating probe tip on the outer tube when the rotating probe tip is in the second position.
